(19) United States
(12) Patent Application Publication
(10) Pub. No.: US 2006/0198863 A1
(43) Pub. Date: Sep. 7, 2006

(54) Ceramic composition for filling bone defects

(75) Inventor: Carl Alexander DePaula, Cranbury, NJ (US)

Correspondence Address: JOHN S. HALE;GIPPLE & HALE, 6665-A OLD DOMINION DRIVE, MCLEAN, VA, 22101, US

(73) Assignee: Musculoskeletal Transplant Foundation

(21) Appl. No.: 11/070,233

(22) Filed: Mar. 3, 2005

(51) Int. Cl. A61K 38/18; A61K 31/728

(52) U.S. Cl. 424/422; 514/12; 514/54

(57) ABSTRACT

The invention is directed toward a formable ceramic composition for application to a bone defect site which comprises a ceramic compound of beta tricalcium phosphate particles. The particle size ranges from about 40 microns to 500 microns and is mixed in a hydrogel carrier containing citric acid buffer, the hydrogel component of the carrier ranging from about 1.0 to 5.0% of the composition and the composition has a pH between 7.0 to 7.8.

### RELATED APPLICATIONS

There are no related applications.

### FIELD OF INVENTION

The present invention is generally directed toward a surgical ceramic bone implant product and more specifically is a moldable and shapeable composition for filling bone defects using ceramics such as tricalcium phosphate, TCP, Ca3(PO4)2, alpha and beta, hydroxyapatite, calcium phosphates and calcium sulfate having a size ranging from 40 to 500 μm with a weight ranging from 66% to 76% by weight of the composition mixed in a fluid carrier having a high molecular weight viscous excipient derived from a hydrogel such as sodium hyaluronate.

### BACKGROUND OF THE INVENTION

Surgical implants should be designed to be biocompatible in order to successfully perform their intended function. Biocompatibility may be defined as the characteristic of an implant acting in such a way as to allow its therapeutic function to be manifested without secondary adverse affects such as toxicity, foreign body reaction or cellular disruption.

Formable compositions are used to correct surgical defects that may be caused by trauma, pathological disease, surgical intervention or other situations where defects need to be managed in osseous surgery. It is important to have the defect filler in the form of a stable, viscous formable composition to facilitate the placement of the composition into the surgical site which is usually uneven in shape and depth The surgeon will take the composition on a spatula or other instrument and trowel it into the site or take it in his/her fingers to shape the bone defect material into the proper configuration to fit the site being corrected. It is also important that the defect filler be biocompatible.

Many products have been developed in an attempt to treat this surgical need for a biocompatible formable material. One such example is autologous bone particles or segments recovered from the patient. When removed from the patient, the segments or bone particles are wet and viscous from the associated blood. This works very well to heal the defect but requires significant secondary surgery resulting in lengthening the surgery, extending the time the patient is under anesthesia and increasing the cost In addition, a significant increase in patient morbidity is attendant in this technique as the surgeon must take bone from a non-involved site in the patient to recover sufficient healthy bone, marrow and blood to perform the defect filling surgery. This leads to significant post-operative pain.

Allograft bone is a logical substitute for autologous bone. It is readily available and precludes the surgical complications and patient morbidity associated with autologous bone as noted above. Allograft bone is essentially a collagen fiber reinforced hydroxyapatite matrix containing active bone morphogenic proteins (BMP) and can be provided in a sterile form. The demineralized form of allograft bone is naturally both osteoinductive and osteoconductive. The demineralized allograft bone tissue is fully incorporated in the patient's tissue by a well established biological mechanism. It has been used for many years in bone surgery to fill the osseous defects previously discussed.

Demineralized allograft bone is usually available in a lyophilized or freeze dried and sterile form to provide for extended shelf life. The bone in this form is usually very coarse and dry and is difficult to manipulate by the surgeon. One solution to use such freeze dried bone has been provided in the form of a gel, GRAFTON®, a registered trademark of Osteotech Inc., which is a simple mixture of glycerol and lyophilized, demineralized bone powder of a particle size in the range of 0.1 cm to 1.2 cm (1000 microns to 12,000 microns) as is disclosed in U.S. Pat. No. 5,073,373.

GRAFTON works well to allow the surgeon to place the allograft bone material at the site. However, the carrier, glycerol has a very low molecular weight (92 Daltons) and is very soluble in water, the primary component of the blood which flows at the surgical site. Glycerol also experiences a marked reduction in viscosity when its temperature rises from room temperature (typically 22° C. in an operating room) to the temperature of the patient's tissue, typically 37° C. This combination of high water solubility and reduced viscosity causes the allograft bone material with a glycerol carrier to be ‘runny’ and to flow away from the site almost immediately after placement; this prevents the proper retention of the bone material within the site as carefully placed by the surgeon.

U.S. Pat. No. 5,290,558 discloses a flowable demineralized bone powder composition using an osteogenic bone powder with large particle size ranging from about 0.1 to about 1.2 cm mixed with a low molecular weight polyhydroxy compound possessing from 2 to about 18 carbons including a number of classes of different compounds such as monosaccharides, disaccharides, water dispersible oligosaccharides and polysaccharides.

U.S. Pat. No. 5,356,629 discloses making a rigid composition in the nature of a bone cement to fill defects in bone by mixing biocompatible particles preferably polymethylmethacrylate coated with polyhydroxyethylmethacrylate in a matrix selected from a group which lists hyaluronic acid to obtain a molded semi-solid mass which can be suitably worked for implantation into bone. The hyaluronic acid can also be utilized in monomeric form or in polymeric form preferably having a molecular weight not greater than about one million Daltons. It is noted that the nonbioabsorbable material which can be used to form the biocompatible particles can be derived from xenograft bone, homologous bone, autogenous bone as well as other materials. The bioactive substance can also be an osteogenic agent such as demineralized bone powder, in addition to morselized cancellous bone, aspirated bone marrow and other autogenous bone sources. The average size of the particles employed is preferably about 0.1 to about 3.0 mm, more preferably about 0.2 to about 1.5 mm, and most preferably about 0.3 to about 1.0 mm. It is inferentially mentioned but not taught that particles having average sizes of about 7,000 to 8,000 microns, or even as small as about 100 to 700 microns can be used. This is simply a cement used for implantation of hip prosthesis and is not used to promote bone growth.

U.S. Pat. No. 6,437,018 issued Aug. 20, 2002 owned by the assignee of the present invention discloses a malleable bone putty and a flowable gel composition for application to a bone defect site to promote new bone growth at the site which comprises a new bone growth inducing compound of demineralized lyophilized allograft bone powder. The bone powder has a particle size ranging from about 100 to about 850 microns and is mixed in a high molecular weight hydrogel carrier contain a sodium phosphate saline buffer, the hydrogel component of the carrier ranging from about 0.75 to 4.5% of the composition and having a molecular weight of about at least 160,000 Daltons. The composition has a pH between 6.8-7.4, contains about 25% to about 35% bone powder and can be additionally provided with BMP's. Another malleable bone putty is disclosed in U.S. Pat. No. 6,030,635, now RE 38,522, issued Feb. 29, 2000.

Another product group involves the use of inorganic materials to provide a matrix for new bone to grow at the surgical site. These inorganic materials include hydroxyapatite obtained from sea coral or derived synthetically. Either form may be mixed with the patient's blood and/or bone marrow to form a gel or a putty. Calcium sulfate or plaster of Paris may be mixed with water to similarly form a putty. Other products within this group include ceramics such as tricalcium phosphate.

The use of ceramic compositions utilizing beta tricalcium phosphate and alpha tricalcium phosphate for bone graft substitutes are well known in the art. These graft materials generally harden in place. U.S. Pat. No. 5,522,893 issued Jun. 4, 1996 discloses a bone filling material which is a combination of tricalcium phosphate and dicalcium phosphate salts that are mixed and react to harden and form hydroxycarbonate apatite after implantation.

U.S. Pat. No. 6,231,607 issued May 15, 2001 is directed toward a solid ceramic composition comprising a β-TCP, hydroxyl apatite and a substantial amount of α-TCP.

U.S. Pat. No. 6,521,246 issued Feb. 18, 2003 is directed toward a shaped body comprising a macro-, meso-, and microporous calcium phosphate and having a pore volume. The calcium phosphate is a β-TCP.

Unfortunately, the prior art TCP compositions tend to harden rather quickly and have short if any shelf life. Thus, the composition has to be mixed at the time of surgery or in a short time period before same.

Accordingly, the prior art as embodied in present ceramic technology is replete with problems and only partially addresses the problems inherent in the correcting surgical defects.

### SUMMARY OF THE INVENTION

The subject formulation is a complex mixture of beta tricalcium phosphate particles and a viscous hydrogel such as sodium hyaluronate together with a citric acid monohydrate.

An inventive aspect of this composition is overcoming the stability and handling problems of beta tricalcium phosphate while preserving a favorable biologic response by controlling the particle size of the ceramic granules and the weight percentage in the composition. The favorable handling characteristics of the ceramic putty are due to the specific particle size ranges used and the narrow range of the percentage of ceramic weight in the composition. Particle sizes that are larger than those of the present invention create a putty with a gritty feel which is unacceptable to a surgeon and cannot be effectively used in a syringe. Smaller particle sizes create a putty with acceptable handling characteristics, however they negatively impact the stability of the putty, causing it to harden in the package. Smaller particles would also create an unfavorable biologic response because the small particles would be absorbed too quickly and lose their efficacy, could cause an immunological response, and could migrate though the lymphatic system.

Another inventive aspect of this device is the use of the citric acid to adjust the pH of the composition. Citric acid is useful because it allows the pH of the composition to be lowered into the physiologic range. In addition, it is believed to help the stability of the composition by chelating calcium. The hardening of the composition in the package (syringe) is likely due to the small particle size used. The small particles have a large surface area and would be more readily soluble. The free solubilized from the ceramic in the device will then precipitate likely forming hydroxyapatite and causing the device to harden in the package. Citric acid is believed to help prevent this by chelating calcium and preventing the precipitation of hydroxyapatite and the hardening of the composition in the package.

It is an object of the invention to utilize a ceramic material having a particle size that is useful to achieve the malleability characteristics which results in easy application while allowing easy insertion into the bone defect area and a round particle shape for improved cell friendliness.

It is an additional object of the invention to use a citric acid solution to present the composition in a state of physiological pH at the wound site by neutralizing the pH.

It is also an object of the invention to create a ceramic defect material which can be easily handled by the physician and does not degenerate when contacting blood flow at the surgical site, is ready to use out of the package and requires no mixing.

It is still another object of the invention to create a ceramic defect material which is stable and has an extended shelf life when packaged.

It is another object of the invention to create a ceramic defect material which uses cellular material such as living cells and cell elements.

It is yet another object of the invention to use a growth factor in the ceramic composition.

It is yet another object of the invention to use an anti-infective agent in the ceramic composition.

It is yet another object of the invention to add a small quantify of silicon to the ceramic composition.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 shows a chart reflecting moles of citric acid required per gram of tricalcium phosphate granules.

### DESCRIPTION OF THE INVENTION

The present invention is directed towards a ceramic beta tricalcium phosphate (β-TCP) particle based composition which is applied to bone defects.

A formable composition utilizing the β-TCP with a useful bulk viscosity has been achieved by using a soluble hydrogel such as hyaluronic acid in the carrier. The balance of the carrier formulation is an aqueous solution and preferably includes the addition of a component, namely, a citric acid which lowers the pH to a physiologic range and stabilizes the composition.

The particle size of ceramic particles when mixed with high molecular weight stable viscosity hydrogel and preferred shape which is rounded or spherical in a suitable carrier produces a space between the granules that is occupied by the hyaluronan with clinically useful bone inducing properties. The formability property permits the surgeon to shape the ceramic composition to exactly fit the surgical defect Manipulation of the lump of formable ceramic composition may be done without it sticking to the gloves of the surgeon, behaving somewhat like a wet clay used in sculpting.

It is an important aspect of the present invention that the implant matrix must remain at the wound site and not be washed away by the flowing blood and other irrigation fluids brought to the site by the healing mechanism. While viscous, the aqueous carrier is a high molecular weight macromolecule held together with water linkages (hydrogen bonds) and is not readily dissolved and washed away by the blood and fluids at the wound site.

Thus, the therapeutic formable ceramic composition will not be dissipated by being washed away and will be present to be osteoconductive while being somewhat osteoinductive.

The amount of ceramic β-TCP is maximized to achieve the optimum balance of osteoconductivity and physical handling properties. Adding too much ceramic matrix may create a gritty or sandy condition in which the composition is not enclosed by the surrounding viscous matrix and the ceramic particles are not held together. The preferred type of ceramic material used in the invention is β-TCP having a particle size ranging from 50 to 500 μm.

The primary role of a carrier is to serve as a delivery vehicle. The bulk viscosity of the carrier achieves the design goal of good handling properties by balancing the molecular weight and concentration of the hydrogel used in the formulation. For example, a very high molecular weight hydrogel would use a lower concentration compared to a formulation in which the hydrogel molecular weight was considerably lower with a higher concentration used to achieve the same bulk viscosity. The nominal composition formulation uses a 660,000 Dalton molecular weight sterile hydrogel (sodium hyaluronate). This hydrogel material is used at a 1-5% concentration in the carrier to achieve the bulk viscosity required for the formulation.

The putty is preferably composed at an optimum 71% (wt %) with a usable weight range of 66% to 76% β-TCP ceramic particles that are 50-150 μm in diameter and a 29 % (wt %) hyaluronan carrier with a usable range of 24% to 34%. The 71% ceramic putty has a pH of 7.6, slightly higher than physiologic pH. Citric acid is preferably added to the formulation to lower the pH to physiologic 7.4 and to provide composition stability. Particles less than 20 microns are undesirable because they can cause an inflammatory response and the small particles can migrate through the lymphatic system.

A putty was created using unsieved β-TCP granules (100-900 μm) at 68% weight with the sodium hyaluronate. It was noted that the putty felt grainy but not dry. The putty was attempted to be loaded into syringes. However, the putty particles separated in the syringe when pressure was applied, jamming the syringe. Very few formulations were able to flow out of a syringe.

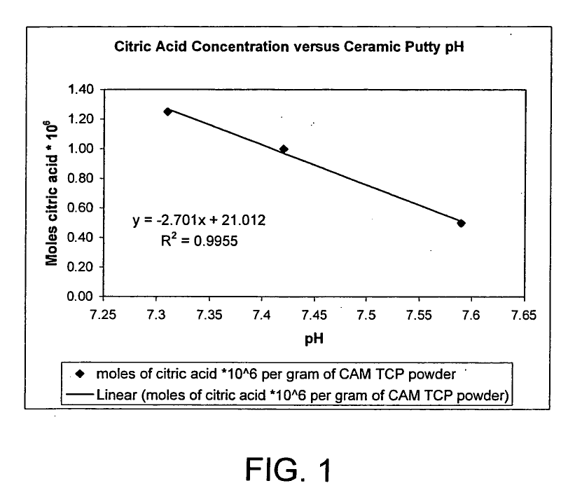

Another putty was then made using β-TCP powder, β-TCP 50-150 μm granules, and β-TCP 150-500 μm granules. In the present formulation, a range of 66% to 76% β-TCP was the preferred range with 71% β-TCP being selected as the optimum ceramic material weight formulation. A one week accelerated aging study was performed at 40° C. (equivalent to 4 weeks at ambient temperatures) on this putty composition and there was no change in the putty. The penetration, pH and handling of the preferred putty did not change from time zero to one week accelerated aging. The average penetration of this formulation was 4.9 mm and the pH was 7.6 without the addition of citric acid. The amount of citric acid required to create a putty with a pH of 7.4 was determined using the chart of FIG. 1.

Materials Used:

1. Beta tricalcium phosphate ceramic (β-TCP) granules, 50-150 μm in diameter.

2. Sodium hyaluronate (NaHy), MW=650,000 to 1,000,000 Daltons, Sorenson's phosphate buffered saline (PBS), pH: 7.4±0.3; Osmolality: 290−340 mOsm/Kg; Viscosity: 1.19×105−1.36×105 cps; Endotoxin:<1.25 EU/ml.

3. Citric acid (1.025*10-6 moles of citric acid monohydrate per gram of TCP powder to the hyaluronan using 10 μL of citric acid solution per gram of TCP powder)

Formulation:

1. Putty with citric acid formulation
1.1. Formulation is 71% (wt %) ranging from 66% to 76% sterile β-TCP powder 50-150 μm sieved powder, sterile 29% (wt %) hyaluronan carrier ranging from 24% to 34% and 10 μL of sterile 0.1025M citric acid solution, per gram of TCP powder.
1.1.1. Weigh hyaluronan (Hy)
1.1.2. Add 1.025*10-6 moles of citric acid monohydrate per gram of TCP powder to the hyaluronan using 10 μL of citric acid solution per gram of TCP powder; mix thoroughly.
1.1.2.1. Make fresh 0.5M citric acid by adding 10.507 g of Citric acid monohydrate to a 100 mL volumetric flask, fill to the line with DI H2O, stir until mixed, pH=1.75 [Citric acid monohydrate, MW=210.14 g/mole]
1.1.2.2. Add 2050 μL of 0.5M citric acid to 7950 μL of DI H2O, and mix to make 10,000 μL of 0.1025M citric acid solution.
1.1.2.3. Add 10 μL of the 0.1025M citric acid solution per gram of TCP powder to the hyaluronan, mix thoroughly.
1.1.3. Add beta tricalcium phosphate (β-TCP) ceramic powder to the Hy at 71%. Mixing a controlled manner by hand, gently because mixing will damage the particles and change the particle size distribution of the particles and this will affect the putty consistency. One stir per second for up to 120 seconds as needed.
1.2. Measure pH, penetration, and handling of each sample at its designated time point and record observations on the handling of the putty.

The natural condition for blood plasma as well as synovial fluid, cerebrospinal fluid, aqueous humor (fluid within the globe of the eye) is at a pH of 7.3-7.4 (reference, Principles of Biochemistry, Chapters 34 & 35; White, Handler and Smith, McGraw Hill, NY, 1964). At very slight changes in pH, blood cells will shift their equilibrium of hemoglobin. This hemoglobin concentration will change over the small pH range of 7.3 to 7.7 (White et al., p. 664). In addition, at significantly lower pH values in the acidic range, protein molecules will denature, i.e., degrade. Thus, it is important to maintain any surgical implant which is intimate contact with blood at a biocompatible condition of about pH 7.2-7.4.

It is important to note that the body has many complex and redundant mechanisms to maintain its biochemical balance. The blood pH can be adjusted by several means to its normal, physiologic pH. Thus the presence of a non-physiologic material at the site of a bleeding bone wound will eventually be overcome and any non-biocompatible condition will return to normal pH. The preferred formulation will start out and maintain pH within the range of 7.0 to 7.8 without stressing the body's biochemical mechanisms when the ceramic composition material is applied at the wound site.

In achieving physiologic pH, the formulation uses a citric acid solution to buffer the sodium hyaluronate viscous hydrogel carrier and maintain pH stability.

The pH is adjusted to the physiologic range of 7.0 to 7.8 pH, preferably 7.2-7.4.

Thus, the invention induces the presence of soluble calcium at the bone defect site. This will encourage new bone growth through the normal biochemical mechanism. Soluble calcium can be attracted to the surgical site by using the citric acid buffer. It is believed that the buffer attracts calcium cations to the site from the surrounding healthy bone and creates an equilibrium concentration of the calcium precisely at the site of healing where it is most desirable to grow new bone.

It is a well known principal of physiology that osmotic pressure must be maintained within a narrow range to assure healthy conditions for the many cell types present in normal or surgically wounded cells. The condition of normal osmotic pressure is referred to as an isotonic state and is quantified in humans by the value of about 300 mOsmol/Kg. The sodium hyaluronate formulation is buffered to isotonic conditions using Sorenson's phosphate buffered saline (PBS) or a carbonate buffer, citrate buffer, or other organic buffer.

Sodium hyaluronate in the form of the sodium salt is generally described as an acid mucopolysaccharide. It is envisioned that suitable amounts of bone morphogenic proteins (BMP) can be added to the putty at any stage in the mixing process to induce accelerated healing at the bone site. BMP directs the differentiation of pluripotential mesenchymal cells into osteoprogenitor cells which form osteoblasts.

The composition with satisfactory formability, shelf life stability and handling properties has a sodium hyaluronate with a molecular weight ranging from 650,000 to 1,000,000 Daltons with the sodium hyaluronate concentration in the carrier ranging from 0.75 to 5.0% with a ceramic beta tricalcium phosphate concentration ranging from 66% to 76% by weight with a particle size of 50 to 150 microns.

Additives which are beneficial to bone growth and which are added into the formable composition are living cells and cell elements such as chondrocytes, red blood cells, white blood cells, platelets, blood plasma, bone marrow cells, mesenchymal stem cells, pluripotential cells, osteoblast, osteoclasts, and fibroblasts, epithelial cells, and endothelial cells. These cells or cell elements or combinations of the same are present at a concentration of 10⁵ to 10⁸ per cc of carrier and are added into the composition at time of surgery.

Growth factor additives which can be used in the present invention either at the time of packaging or at surgery depending on the stability of the growth factor are transforming growth factor (TGF-beta), insulin growth factor (IGF-1); platelet derived growth factor (PDGF), vascular endothelial growth factor (VEGF), fibroblast growth factor (FGF) (numbers 1-23), osteopontin, growth hormones such as somatotropin cellular attractants and attachment agents and bone morphogenic proteins (BMP's).

Any number of medically useful substances can be used in the invention by adding the substances to the composition at any steps in the mixing process or directly to the final composition. Such substances include Type I collagen and insoluble collagen derivatives for blood vessel formation and/or bone formation, hydroxyapatite, and soluble solids and/or liquids dissolved therein.

Also included in the additives which may be added to the carrier are antiviricides such as those effective against HIV and hepatitis; antimicrobial and/or antibiotics such as erythromycin, bacitracin, neomycin, penicillin, polymyxin B, tetracycline, viomycin, chloromycetin and streptomycin, cefazolin, ampicillin, azactam, tobramycin, clindamycin and gentamycin.

It is also envisioned that other additives which can be added are amino acids, peptides, vitamins, co-factors for protein synthesis; hormones; endocrine tissue or tissue fragments; synthesizers; enzymes such as collagenase, peptidases, oxidases; polymer cell scaffolds with parenchymal cells; angiogenic drugs and polymeric carriers containing such drugs; collagen lattices; biocompatible surface active agents, antigenic agents; cytoskeletal agents; cartilage fragments.

Other additives for the putty comprise adding 0.8 wt % silicon for bone growth stimulation or adding a carbonate substituted apatite (bone apatite). Food (glucose) for the cells could be added along with amino acids. It is also envisioned that one could add additional glycosaminoglycans (GAGs) or proteoglycans to further improve and speed bone formation (the specific GAGs of physiological significance are hyaluronic acid, dermatan sulfate, chondroitin sulfate, heparin, heparan sulfate, and keratan sulfate). In addition, carboxymethylcellulose could be added to the formulation to provide additional elasticity to the putty for improved handling or other surgical applications.

In the following examples the weight of the various carrier components used is as follows:

### EXAMPLE 1

### Unacceptable Putty Formulation

Mix 1.162 g of β-TCP granules (granules 50-150 μm in diameter, β-TCP granules are round, spherical, and uniform upon microscopic examination) with 0.845 g of NaHy, to create a 58% by weight TCP putty. This putty sample was too wet, did not hold its shape, and is not useful.

### EXAMPLE 2

### Unacceptable Putty Formulation

Mix 1.162 g of β-TCP granules (β-TCP granules, 50-150 μm, with irregularly shaped granules upon microscopic examination, with 20% of the particles less than 50 μm) with 0.845 g of NaHy, to create a 58% by weight TCP putty. This putty is not wet or sticky and it holds its shape, and initially appeared useful. At time zero, it has a penetration of 4.65 mm, and a pH of 8.95. At time 24 hours, it has a penetration of 4.28 mm, and a pH of 9.83, above the physiologic range. At time 3 weeks, it has a penetration of 6.28 mm, and a pH of 5.9. However, after sitting in its packaging for 3 weeks it became hardened and was no longer moldable.

### EXAMPLE 3

### Acceptable Putty Formulation

Mix 28.401 g of β-TCP (granules 50-150 μm in diameter, β-TCP granules are round, spherical, and uniform upon microscopic examination) with 11.601 g of NaHy, to create a 71% by weight TCP putty. This forms a putty that is not too wet or too dry and it is moldable and shapeable. At time zero, it had a mean penetration of 3.35 (sd=0.681) mm, and a pH of 7.57 (sd=0.01). At time of one week accelerated aging at 40° C., it had a mean penetration of 3.53 (sd=0.590) mm, and a pH of 7.71 (sd=0.005). This is apparently because the TCP dissolved to change the pH of the composition. The handling properties did not changed after one week. The putty molds well and holds preformed shapes, it is not very sticky. The bright white color is good. It has no odor.

### EXAMPLE 4

### Acceptable Putty Formulation With Citric Acid

Measure 2.4532 g of NaHy suspension and add 60 μL of 0.1M citric acid monohydrate and mix thoroughly. Add 6.0022 g of β-TCP (granules 50-150 μm in diameter, β-TCP granules are round, spherical, and uniform upon microscopic examination) to the mixture and mix gently, to create a 71% by weight putty data. The pH at time zero was 7.42 (0.01) and the penetration was 4.95 mm (0.06). This putty has handling properties the same as Example 3 was stable and maintained low pH.

### EXAMPLE 5

### Acceptable Putty Formulation With Citric Acid

Mix 28.405 g of β-TCP (granules 50-150 um in diameter, β-TCP granules are round, spherical, and uniform upon microscopic examination) with 11.613 g of NaHy, to create a 71% by weight TCP putty with 0.15M citric acid. This forms a putty that is not too wet or too dry and it is moldable and shapeable. At time zero, it has a mean penetration of 5.03 (sd=11.89) mm, and a pH of 7.16 (sd=0.01). After one week accelerated aging at 40° C., it has a mean penetration of 4.27 mm (sd=0.590) mm, and a pH of 7.2 (sd=0.005). The handling properties did not change after one week. The putty molds well and holds preformed shapes, it is not very sticky. The bright white color is good.

### EXAMPLE 6

### Unacceptable Putty Formulation

Mix 1.56 g of β-TCP granules (granules 50-150 μm in diameter, β-TCP granules are round, spherical, and uniform upon microscopic examination) with 0.44 g of NaHy, to create a 78% by weight TCP putty. This putty sample was too dry, crumbly, does not hold together, or sustain physical integrity.

In Examples 3-5, living cells and cell elements such as chondrocytes, red blood cells, white blood cells, platelets, blood plasma, bone marrow cells, mesenchymal stem cells, pluripotential cells, osteoblasts, osteoclasts, and fibroblasts, epithelial cells, and endothelial cells can be added into the composition. These cells or cell elements or combinations of the same are present at a concentration of 10⁵ to 10⁸ per cc of carrier and are added into the composition at time of surgery.

Similarly growth factor additives can be used in the present composition either at the time of packaging or at surgery depending on the stability of the growth factor are transforming growth factor (TGF-beta), insulin growth factor (IGF-1); platelet derived growth factor (PDGF), vascular endothelial growth factor (VEGF), fibroblast growth factor (FGF) (numbers 1-23), osteopontin, growth hormones such as somatotropin cellular attractants and attachment agents. Fiberblast growth factor is added in the amount of 2 to 4 milligrams in 10 cc of carrier solution.

The mixing of the ceramic β-TCP powder into a sterile hydrogel solution is undertaken in a sterile chamber. The mixed formable ceramic composition is then placed in a sterile container such as an impervious syringe barrel or vial, sealed and placed in a sterile sealed package to which stable growth factors are added with the cell material and unstable growth factors added to the composition at the time of surgery.

One process commonly used to achieve sterility is sterile filtration of the sodium hyaluronate followed by aseptic mixing of the ceramic and sodium hyaluronate. Another method is to irradiate the sodium hyaluronate material first and then continue with aseptic mixing of the ceramic. Irradiation sources of either electron beam or gamma (Cobalt 60 isotope) are commercially available. The ceramic is commonly sterilized using gamma radiation.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A sterile formable ceramic composition for application to a bone defect site comprising ceramic tricalcium phosphate particles in an aqueous carrier solution, the ceramic particles being added to a viscous carrier at a concentration ranging from about 66% to about 76% (w/w), the carrier comprising a hydrogel component of sodium hyaluronate or its derivatives in a buffered phosphate solution with citric acid, said hydrogel ranging from about 1.0% to about 5.0% by weight of the aqueous carrier solution and said hydrogel component having a high molecular weight ranging from about six hundred fifty thousand to one million Daltons with a stable viscosity at a temperature ranging from about 22° C. to about 37° C. and said composition having a pH ranging from about 7.0 to about 7.8

2. A sterile formable ceramic composition as claimed in claim 1 wherein said ceramic particles are β-TCP ranging in size from 50 microns to 150 microns at a concentration ranging from about 69% to 73% by weight of the composition.

3. A sterile formable ceramic composition as claimed in claim 1 wherein said ceramic particles are β-TCP ranging in size from 50 microns to 150 microns at a concentration ranging from about 69% to 73% by weight of the composition.

4. A sterile formable ceramic composition as claimed in claim 1 including growth factor additive added to said composition, said growth factor comprising one or more of a group consisting of transforming growth factor (TGF-beta), insulin growth factor (IGF-1); platelet derived growth factor (PDGF), vascular endothelial growth factor (VEGF), fibroblast growth factor (FGF) (numbers 1-23), osteopontin, growth hormones such as somatotropin cellular attractants and attachment agents.

5. A sterile formable ceramic composition as claimed in claim 1 including growth factor additive added to said composition comprising one or more of a group consisting of fibroblast growth factor (FGP) (numbers 1-23) in the amount of 2-4 milligrams in 10 cc of carrier solution.

6. A sterile formable ceramic composition as claimed in claim 1 wherein said composition includes an additive citric acid.

7. A sterile formable ceramic composition as claimed in claim 1 wherein said composition includes the addition of Type I collagen.

8. A sterile formable ceramic composition as claimed in claim 1 wherein said composition includes the addition of a growth factor additive added to said composition, said growth factor comprising one or more of a group consisting of transforming growth factor (TGF-beta), insulin growth factor (IGF-1); platelet derived growth factor (PDGF), vascular endothelial growth factor (VEGF), fibroblast growth factor (FGF) (numbers 1-23), osteopontin, growth hormones such as somatotropin cellular attractants and attachment agents.

9. A sterile formable ceramic composition as claimed in claim 1 wherein said composition includes the addition of antiviricides such as those effective against HIV and hepatitis; antimicrobial and/or antibiotics such as erythromycin, bacitracin, neomycin, penicillin, polymyxin B, tetracycline, viomycin, chloromycetin and streptomycin, cefazolin, ampicillin, azactam, tobramycin, clindamycin and gentamycin.

10. A sterile formable ceramic composition for application to a bone defect site to comprising ceramic beta tricalcium phosphate particles ranging in size from 50 to 150 microns in an aqueous carrier solution, the ceramic particles being added to a viscous carrier at a concentration ranging from about 66% to about 76% (w/w), the carrier comprising a hydrogel component of sodium hyaluronate or its derivatives in a buffered sodium phosphate solution with added citric acid aqueous solution, said hydrogel ranging from about 1.0% to about 5.0% by weight of the aqueous carrier solution and said hydrogel component having a high molecular weight ranging from about six hundred fifty thousand to one million Daltons with a stable viscosity at a temperature ranging from about 22° C. to about 37° C., said composition having a pH ranging from about 7.0 to about 7.8.

11. A formable ceramic composition as claimed in claim 10 including a cellular material taken from a group consisting of living cells and cell elements such as chondrocytes, red blood cells, white blood cells, platelets, blood plasma, bone marrow cells, mesenchymal stem cells, pluripotential cells, osteoblasts, osteoclasts, and fibroblasts, epithelial cells, and endothelial cells, these cells or cell elements or combinations of the same are present at a concentration of 10⁵ to 10⁸ per cc of the carrier.

12. A sterile formable ceramic composition as claimed in claim 10 wherein said composition includes the addition of Type I collagen.

13. A sterile formable ceramic composition as claimed in claim 10 wherein said composition includes the addition of a growth factor additive added to said composition, said growth factor comprising one or more of a group consisting of transforming growth factor (TGF-beta), insulin growth factor (IGF-1); platelet derived growth factor (PDGF), vascular endothelial growth factor (VEGF), fibroblast growth factor (FGF) (numbers 1-23), osteopontin, growth hormones such as somatotropin cellular attractants and attachment agents.

14. A sterile formable ceramic composition as claimed in claim 10 wherein said composition includes the addition of antiviricides such as those effective against HIV and hepatitis; antimicrobial and/or antibiotics such as erythromycin, bacitracin, neomycin, penicillin, polymyxin B, tetracycline, viomycin, chloromycetin and streptomycin, cefazolin, ampicillin, azactam, tobramycin, clindamycin and gentamycin.

15. A sterile formable ceramic composition as claimed in claim 10 including growth factor additive added to said composition comprising one or more of a group consisting of fibroblast growth factor (FGF) (numbers 1-23) in the amount of 2-4 milligrams in 10 cc of carrier solution.

16. A sterile formable ceramic composition for application to a bone defect site to comprising β-TCP particles in an aqueous carrier solution containing citric acid and a sodium hyaluronate ranging from about 1.0% to 5.0% by weight of the aqueous carrier, the ceramic particles ranging from 50 to 150 microns being added to a viscous carrier at a concentration ranging about 71% (w/w), said composition having a pH after aging between about 7.2 to about 7.4.

17. A sterile formable ceramic composition as claimed in claim 16 including a cellular material additive taken from a group consisting of living cells and cell elements such as chondrocytes, red blood cells, white blood cells, platelets, blood plasma, bone marrow cells, mesenchymal stem cells, pluripotential cells, osteoblasts, osteoclasts, and fibroblasts, epithelial cells, and endothelial cells. These cells or cell elements or combinations of the same are present at a concentration of 10⁵ to 10⁸ per cc of the carrier

18. A sterile formable ceramic composition as claimed in claim 16 including growth factor additive added to said composition, said growth factor comprising one or more of a group consisting of transforming growth factor (TGF-beta), insulin growth factor (IGF-1); platelet derived growth factor (PDGF), vascular endothelial growth factor (VEGF), fibroblast growth factor (FGF) (numbers 1-23), osteopontin, growth hormones such as somatotropin cellular attractants and attachment agents.

19. A sterile formable ceramic composition as claimed in claim 16 including growth factor additive added to said composition comprising one or more of a group consisting of fibroblast growth factor (FGF) (numbers 1-23) in the amount of 2-4 milligrams in 10 cc of carrier solution.

20. A sterile formable ceramic composition as claimed in claim 16 wherein said composition includes the addition of Type I collagen.

21. A sterile formable ceramic composition as claimed in claim 16 wherein said composition includes the addition of a growth factor additive added to said composition, said growth factor comprising one or more of a group consisting of transforming growth factor (TGF-beta), insulin growth factor (IGF-1); platelet derived growth factor (PDGF), vascular endothelial growth factor (VEGF), fibroblast growth factor (FGF) (numbers 1-23), osteopontin, growth hormones such as somatotropin cellular attractants and attachment agents.

22. A sterile formable ceramic composition as claimed in claim 16 wherein said composition includes the addition of antiviricides such as those effective against HIV and hepatitis; antimicrobial and/or antibiotics such as erythromycin, bacitracin, neomycin, penicillin, polymyxin B, tetracycline, viomycin, chloromycetin and streptomycin, cefazolin, ampicillin, azactam, tobramycin, clindamycin and gentamycin.

23. A sterile formable ceramic composition as claimed in claim 16 wherein said composition includes the addition of about 0.8 wt % silicon.

24. A sterile formable ceramic composition as claimed in claim 16 wherein said composition includes the addition of BMP's.
